(19) United States
(12) Patent Application Publication
(10) Pub. No.: US 2007/0197797 A1
(43) Pub. Date: Aug. 23, 2007

(54) Compounds and methods for carbazole synthesis

(76) Inventor: Peter J. Harrington, Louisville, CO (US)

Correspondence Address: Roche Colorado Corporation, 2075 North 55th Street, Boulder, CO, 80301, US

(21) Appl. No.: 11/646,830

(22) Filed: Dec. 28, 2006

Related U.S. Application Data
(60) Provisional application No. 60/755,438, filed on Dec. 30, 2005.

(51) Int. Cl. C07D 209/82

(52) U.S. Cl. 548/444

(57) ABSTRACT

Compounds having bi-cyclic structure comprising a partially unsaturated 6-carbon first cyclic moiety interconnected to a 6-carbon second cyclic moiety second via a divalent linking moiety are provided. The compounds can be used as intermediates compounds in methods for the synthesis of carbazoles and derviatives thereof, including carvedilol, and tricyclic alkylhydroxamates, which do not require Fischer indole synthetic steps. Methods of preparing the compounds having bi-cyclic structures are also provided.

### CROSS-REFERENCE TO RELATED APPLICATION

The present non-provisional patent Application claims priority under 35 USC §119(e) from U.S. Provisional Patent Application having Ser. No. 60/755,438, filed on Dec. 30, 2005, and titled COMPOUNDS AND METHODS FOR CARBAZOLE SYNTHESIS, wherein the entirety of said provisional patent application is incorporated herein by reference.

### FIELD OF THE INVENTION

The present invention is related to the synthesis of carbazoles. The present invention is also related to compounds associated with the synthesis of carbazoles.

### BACKGROUND OF THE INVENTION

Biologically active compounds that include an indole ring structure have been shown to be useful for the treatment of a variety of medical conditions. These compounds include indoleamines such as melatonin, carbazoles such as carvedilol, tricyclic alkylhydroxamates, and carbolines such as tetrahydrocarbolines, pyrimidoindoles, and vinpocetine.

The carbazole carvedilol is known as a member of a class of compounds commonly referred to as beta-blockers, which affect the heart and circulatory system and which are often used to treat hypertension. The pharmacological activity of carvedilol is of a nonselective beta-adrenoreceptor antagonist and an alpha₁-adrenoreceptor antagonist. In other words, carvedilol blocks the molecular receptors of the adrenergic nervous system and reduces the heart rate and contraction force. Carvedilol also blocks adrenergic receptors on arteries causing arteriole relaxation and a drop in blood pressure. This feature offers unique benefits for hemodynamic balance in hypertension, heart failure, and ischemic heart disease. Carvedilol also has anti-oxidant and anti-proliferative properties that further differentiate it from other β-blocking agents. Various synthetic schemes have been used to prepare carvedilol (see, for example, U.S. Pat. Nos. 4,503,067, 5,786,356, 6,140,352, 6,514,968, 6,699,997, and 6,730,326). Indole derivatives such as carbazolone, carbazole, and related compounds are important intermediate compounds in the synthesis of carvediol and other related compounds.

Carbazole derivatives such as 2- and 4-hydroxycarbazoles are also important intermediate components in the synthesis of a class of cell proliferation inhibitors as described in International Publication No. WO 02/085883. These cell proliferation inhibitors are tricyclic alkylhydroxamates that have histone deacylase (HDAC) inhibitor activity. These compounds can be used in a method for treating the proliferation of malignant cells, and are thought to be useful in the treatment of cancer.

As shown in FIG. 1, the synthesis of carvedilol and some tricyclic alkylhydroxamates typically involves one or more steps (Rxn A) that lead to the formation of 1,2,3,4a,9,9a-tetrahydro-carbazol-4-one (schematically denoted as Compound A), and then the dehydrogenation (Rxn B) of Compound A to provide 4-hydroxycarbazole (Compound B):

[image]

Commonly used approaches (i.e., Rxn A) leading to the formation of Compound A involve Fisher indole synthetic steps. For example, as shown in FIG. 2, preparation of Compound A may be accomplished by the reaction of cyclohexane-1,3-dione (Compound C) with phenylhydrazine (Compound D) to obtain a hydrazone as represented by Compound E (3-(phenyl-hydrazono)-cyclohexanone):

[image]

For various reasons the conversion of the hydrazone (Compound E) to the carbazolone (Compound A) via reaction A (the Fisher indole synthetic step) is not ideal. First, this approach calls for the use of significant quantities of acid. This is generally undesirable as it generates significant acid by-products, as well as other by-products such as heavy metals, sulfates, or phosphates. The presence of these by-products can impose additional precautions during synthesis. These precautions are amplified when the production of a product is desired at a large scale.

In addition, Fisher indole synthesis can be inefficient. For example, tetrahydro-4-oxocarbazole is typically only produced at about a 50% yield when Fisher indole synthetic steps are employed.

Furthermore, in some cases the regioselectivity of the reaction is rather poor when unsymmetrical substrates are used, such as unsymmetrical ketones. This can lead to a decrease in yield of the desired reaction product, and overall make the process less efficient.

The regioselectivity is also affected by the choice of acid, solvent, and temperature of the reaction. Therefore, particular reaction conditions that are less desired for reasons such as reagent cost, time, and reaction conditions, may be required in the case of Fisher indole synthetic processes which are often used for the preparation of carbazolone, carbazole, and related compounds.

### SUMMARY OF THE INVENTION

The present invention provides novel methodologies and compounds useful for the synthesis of carbazoles, including carbazolones and hydroxycarbazoles. The inventive methods and compounds described herein can be used for the synthesis of carbazole derviatives, such as carvedilol, and tricyclic alkylhydroxamates as described in International Publication No. WO 02/085883.

One aspect of the invention provides compounds having a bi-cyclic structure (that is, a compound having two carbon-containing ring structures). These compounds having a bi-cyclic structure can be formed as intermediates in the synthesis of a carbazole. In particular, these intermediate compounds can be oxidized and cyclized to form a carbazolone, the reaction which advantageously does not require the use of excess amounts of strong acids (which is common in Fischer indole synthetic steps). In this regard, the benefits of the present inventive methods and compounds are apparent.

The bi-cyclic structure comprises a 6-carbon first cyclic moiety having at least one carbon-carbon double bond (e.g., at least partially unsaturated). The first cyclic moiety also comprises a keto group or a hydroxyl group bonded to a cyclic carbon of the first cyclic moiety. The at least one carbon-carbon double bond is present between the alpha and beta carbons (the alpha and beta carbons defined on the first cyclic moiety by the keto group or the cyclic carbon that is bonded to the hydroxyl group) of the cyclic backbone. The bi-cyclic structure also comprises a 6-carbon second cyclic moiety having no carbon-carbon double bonds in the cyclic backbone and comprising a hydroxyl group bonded to a cyclic carbon. The cyclic carbon in the beta position on the first cyclic moiety is bonded via a divalent linking moiety, to the cyclic carbon in the alpha position on the second cyclic moiety (the alpha carbon defined on the second cyclic moiety by the cyclic carbon that is bonded to the hydroxyl group). Preferably the divalent linking moiety includes an N or O atom.

The bi-cyclic compound can individually include single or multi-atom substituent(s) bonded to one or more of the cyclic carbons. Preferably, the substituents (as represented by R₁ and, in some cases, R₂ groups in the formulas provided herein) are, individually, groups that are specifically non-reactive under oxidative conditions useful for converting a compound having a bi-cyclic structure to a compound having a fused tricyclic structure. For example, oxidation of the hydroxyl group on the second cyclic moiety leads to the formation of a keto intermediate and cyclization of the compound of formula I. These groups are herein referred to as “oxidatively nonreactive groups.” In some preferred aspects R₁ and R₂ and are individually selected from H and linear, branched, or cyclic alkyl groups, and most preferably from H and C₁-C₄ alkyl groups.

In some aspects, a compound of the invention having the bi-cyclic structure (as described) is provided by a compound of formula I: 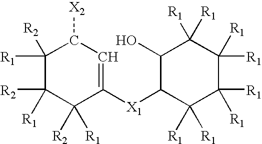
wherein X₁ is a divalent linking moiety, preferably O or NR₃, and R₃ is a single or multi-atom group, preferably H or C₁-C₄ alkyl; and C—X₂ is either C═O or C—OH, wherein if C—X₂ is C═O then R₁ and R₂ are independently selected from single and multi-atom groups, or if C—X₂ is C—OH then R₂ is zero and R₁ is independently selected from single and multi-atom groups. In some aspects R₁ and R₂ are independently selected from oxidatively nonreactive groups, for example, preferably R₃ is H or C₁-C₄ alkyl, and if C—X₂ is C═O, R₁ and R₂ are preferably independently selected from H and C₁-C₄ alkyl, or if C—X₂ is C—OH then R₂ is zero and R₁ is independently selected from H and C₁-C₄ alkyl.

In some specific aspects, in the compound of formula I, X₁ is NR₃, C—X₂ is C═O, and one or more of R₁ and R₂ are H. For example, in these aspects the compound of formula I can be 3-(2-hydroxy-cyclohexylamino)-cyclohex-2-enone:

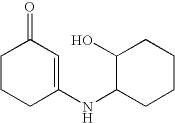

In other specific aspects, in the compound of formula I, X₁ is O, C—X₂ is C═O, and more of R₁ and R₂ are H. For example, in these aspects the compound of formula I can be 3-(2-hydroxy-cyclohexyloxy)-cyclohex-2-enone:

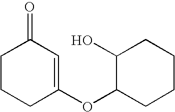

Compounds of formula I can be subjected to oxidation and cyclization. A compound produced by oxidation and cyclization is a carbazolone, such as 1,2,3,5,6,7,8,9-Octahydro-4H-carbazol-4-one (OHOC; Compound I), which can represent another intermediate compound in carbazole synthesis. Dehydrogenation of the carbazolone can provide a carbazole, such as 4-hydroxycarbazole (Compound B).

Oxidation and cyclization of compounds of formula I can advantageously be carried out by a palladium-catalyzed reaction, providing good yields of the carbazolone. In addition, the palladium catalyst can be readily separated from the reaction mixture and also regenerated for subsequent use, thereby presenting further processing and economic benefits. These advantages are in addition to formation of the carbazolone and hydroxycarbazole not requiring use of a strong acid, as would otherwise be associated with Fisher indole synthetic steps.

The methods of the invention have been shown to produce compounds of formula I having excellent yields, such as greater than 98%.

Another aspect of the invention provides methods for the synthesis of compounds of formula I. These methods can also be used in a synthetic scheme for the synthesis of carbazoles and derivatives thereof.

In one aspect, the method comprises a synthetic step of reacting a first compound having a 6-carbon cyclic structure comprising a keto group with a second compound having a 6-carbon cyclic structure. In this method either (a) the first compound comprises a primary amine group bonded to a cyclic carbon and the second compound comprises an oxygen bonded to a cyclic carbon that is reactive with the primary amine, or (b) the second compound comprises a primary amine group bonded to a cyclic carbon and the first compound comprises an oxygen bonded to a cyclic carbon that is reactive with the primary amine.

In this aspect, the first compound can be represented by a compound of formula II: 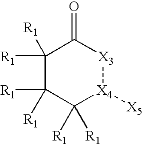
and the second compound can be represented by a compound of formula III: 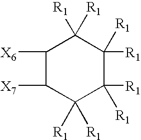
where, in both formula II and formula III, R₁ is independently selected from single and multi-atom groups and either (a) in formula II X₃—X₄—X₅ is C═C—NR₂H, wherein R₂ is a single or multi-atom group, and in formula III X₆ and X₇ form an oxide ring; or (b) in formula II X₃—X₄—X₅ is C—C═O and in formula III X₆ is OH and X₇ is NR₂H wherein R₂ is a single or multi-atom group.

In some aspects R₁ and R₂ are independently selected from oxidatively non-reactive groups. Preferably, R₁ is independently selected from H and C₁-C₄ alkyl groups, and in both C═C—NR₂H and NR₂H R₂ is H or C₁-C₄ alkyl groups.

In some specific aspects of (a) the first compound is 3-amino-2-cyclohexene-1-one and the second compound is cyclohexene oxide. In some specific aspects of (b) the first compound is 1,3-cyclohexanedione and the second compound is 2-aminocyclohexanol.

Synthesis can be performed using equimolar amount of compounds of II and III in a non-polar (e.g., toluene) solvent system at temperatures of greater than 100° C.

In another aspect for the synthesis of compounds of formula I, the invention provides a method comprising a synthetic step of reacting a first compound comprising a 6-carbon cyclic moiety having no carbon-carbon double bonds and comprising two keto groups with a second compound comprising a 6-carbon cyclic moiety having no carbon-carbon double bonds and comprising two hydroxyl groups bonded to cyclic carbons.

In this aspect, the first compound can be represented by a compound of formula IV: 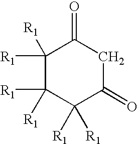
and the second compound can be represented by a compound of formula V: 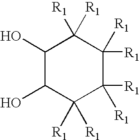
where, in both formula IV and formula V, R₁ is independently selected from single and multi-atom groups. In some aspects R₁ is independently selected from oxidatively non-reactive groups, and is preferably independently selected from H and C₁-C₄ alkyl groups.

In some specific aspects, the first compound is cyclohexanedione and the second compound is cyclohexanediol.

In another aspect, a compound comprising the bi-cyclic structure is provided by a compound of formula VI: 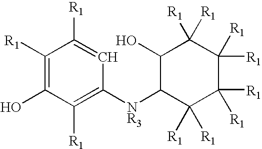
wherein R₁ and R₃ are independently selected from single and multi-atom groups. In some aspects R₁ and R₃ are independently selected from oxidatively nonreactive groups, for example, preferably R₁ and R₃ are independently selected from H and C₁-C₄ alkyl groups.

Compounds of formula VI can be subjected to oxidation and cyclization. A compound produced by oxidation and cyclization is a carbazolone, such as 2-hydroxy-5,6,7,8-tetrahydrocarbazole (Compound K; Example 7), which can represent another intermediate compound in carbazole synthesis. Dehydrogenation of the carbazolone can provide a carbazole, such as 2-hydroxycarbazole (Compound L; Example 7).

In another aspect, the synthesis of a compound of the invention comprising the bi-cyclic structure comprises a synthetic step of reacting a first compound having a 6-carbon cyclic structure and at least one carbon-carbon double bond comprising a hydroxyl group bonded to a cyclic carbon and an amine group bonded to a cyclic carbon with a second compound having a 6-carbon cyclic structure having no carbon-carbon double bonds and comprising a reactive oxygen bonded to one or more cyclic carbons. In a preferred aspect, this method is used to prepare a compound of formula VI.

In this method, and in some aspects, the first compound can be represented by a compound of formula VII: 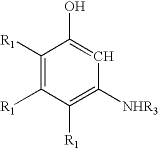
where R₁ is independently selected from single and multi-atom groups and R₃ is also a single or multi-atom group. In some aspects R₁ and R₃ are independently selected from oxidatively non-reactive groups, and preferably R₁ is independently selected from H and C₁-C₄ alkyl, and R₃ is H or C₁-C₄ alkyl; and the second compound can be represented by a compound of formula III: 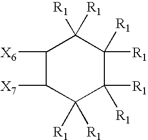
wherein R₁ is independently selected from single and multi-atom groups, preferably independently selected from oxidatively non-reactive groups such as H and C₁-C₄ alkyl, and wherein X₆ and X₇ form an oxide ring.

In some specific aspects, the first compound is 3-amino-phenol and second compound is cyclohexene oxide.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Generally, the invention provides compounds comprising a bi-cyclic structure and methods for forming these compounds, as well as using these compounds or methods in the synthesis of a carbazole or derivative thereof. The bi-cyclic structure comprises a first 6-carbon cyclic moiety having at least one carbon-carbon double bond and a second 6-carbon cyclic moiety. The first and second moieties are interconnected via a divalent linking moiety. The divalent linking moiety can comprises an N or O atom. The first and second cyclic moieties also respectively include one or more oxygen-containing groups bonded to cyclic carbons.

In more specific aspects, the bi-cyclic structure comprises a first 6-carbon cyclic moiety having at least one carbon-carbon double bond (e.g., partially unsaturated) and comprising a ketone group or a hydroxyl group bonded to a cyclic carbon of the cyclic backbone, and a 6-carbon second cyclic moiety having no carbon-carbon double bonds and comprising a hydroxyl group bonded to a cyclic carbon of the cyclic backbone, wherein a cyclic carbon in the beta position (as defined by the keto group or the cyclic carbon bonded to the hydroxyl group) on the first cyclic moiety is bonded via a divalent linking moiety to a cyclic carbon in the alpha position (as defined by the cyclic carbon bonded to the hydroxyl group) on the second cyclic moiety. A carbon-carbon double bond is present between the alpha and beta carbons of the first cyclic moiety.

Given the first cyclic moiety can have at least one double bond, in some aspects the first cyclic moiety has a cyclohex-ene base structure. More specifically, for example, the presence of a ketone group can provide the first cyclic moiety with a cyclohex-2-enone base structure.

The first cyclic moiety can also have more than one carbon-carbon double bond. In these cases, the first cyclic moiety has a delocalized electron system in the ring, such as a benzene base structure. In these cases, the requirements for the first cyclic moiety having at least one of the carbon-carbon double bonds between the alpha and beta carbons is met. A first cyclic moiety having more than one carbon-carbon double bond and a hydroxyl group bonded to a cyclic carbon is exemplified by a phenol base structure.

For the second cyclic moiety, the presence of a hydroxyl group can provide a cyclohexanol base structure.

In addition to the one or more oxygen-containing groups bonded to cyclic carbons, the first and second 6-carbon cyclic moieties may include one or more other substituents that are independently selected from single or multi-atom chemical groups. The single or multi-atom chemical groups can also be bonded to other single or multi-atom chemical groups, if present in the compound. Such bonding may form other cyclic structures fused to either, or both, the first and second cyclic moieties.

In some aspects, these other single or multi-atom groups may be selected so as to be non-reactive under the conditions used for conversion of a compound of formula I to a compound having a fused tricyclic structure. For example, the substituents may be non-reactive in the presence of a palladium catalyzed oxidation reaction using halogenated aromatic hydrocarbon as the oxidant. Such groups are referred to herein as “oxidatively non-reactive groups.”

With these concerns in mind, examples of substituents that may be present can be independently selected from hydrogen; linear, branched, or cyclic alkyl; alkoxy, aryl, combinations of these and the like. Hydrogen and lower alkyl of 1 to 4 carbon atoms are most preferred. For example, R₁ and R₂ groups can be independently selected from and include H, methyl, ethyl, propyl, isopropyl, butyl, isobutyl, sec-butyl and tert-butyl.

In one aspect, the present invention relates to compounds of formula I, wherein the first cyclic moiety is represented by (a) and the second cyclic moiety is represented by (b). For purposes of describing the compounds of the invention, the numbering of the cyclic carbons is shown, the numbering of which can be applied to describe compounds having a bi-cyclic structure: 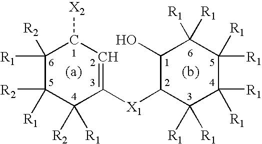
wherein X₁ comprises a divalent linking moiety, and preferably is O or NR₃, wherein R₃ is a single or multi-atom group; and C—X₂ is either C═O or C—OH, wherein if C—X₂ is C═O then R₁ and R₂ are independently selected from single and multi-atom groups, or if C—X₂ is C—OH then R₂ is zero and R₁ is independently selected from single and multi-atom groups. When R₂ is zero the first cyclic structure (a) comprises an aryl moiety.

Preferred compounds of formula I include those wherein each R₁, R₂, and R₃ is independently selected from oxidatively non-reactive groups, and preferably independently selected from H and C₁-C₄ alkyl.

Compounds of formula I, as well as any other compound of any one of the formulas described herein, can be provided in the form of a salt, a racemate, a solvate, a tautomer, or optical isomer thereof, or the like, as desired.

In other aspects, one, or more than one, R₁ group can include reactive substituents. R₁ bearing reactive substituents can be useful if it is desired to include other chemical moieties at one or more locations on a compound of the invention, or a product or derivative thereof. In some cases, an R₁ group is reactive under conditions other than oxidation conditions, other than dehydrogenations conditions, or other than both. A chemical moiety can be added before or after oxidation, cyclization, and dehydration.

Compounds of formula I may include one or more chiral carbons. Whether a given carbon at a position in a compound of formula I may depend on, individually, the R₁ substituent, and in some cases the group as defined by C—X₂.

In some embodiments, and wherein C—X₂ is C═O, the invention provides compounds of formula VIII: 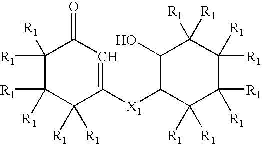
wherein X₁ comprises a divalent linking moiety, and preferably is O or NR₃, wherein R₁ and R₃ are independently selected from single and multi-atom groups, more preferably R₁ and R₃ are independently selected from oxidatively non-reactive groups, and most preferably R₁ and R₃ are independently selected from H and C₁-C₄ alkyl.

In some aspects, the invention provides compounds of formula IX: 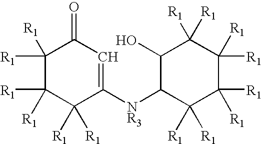
wherein R₁ and R₃ are independently selected from single and multi-atom groups, more preferably R₁ and R₃ are independently selected from oxidatively non-reactive groups, and most preferably R₁ and R₃ are independently selected from H and C₁-C₄ alkyl.

Preferred compounds of the present invention include those wherein R₁ is H and R₃is H.

These preferred compounds can have chiral carbons at least positions 1 and 2 of the second cyclic moiety of the compound of formula IX. Therefore, exemplary compounds of formula IX include 3-((R)2-hydroxy-(S)cyclohexylamino)-cyclohex-2-enone, 3-((S)2-hydroxy-(R)cyclohexylamino)-cyclohex-2-enone, 3-((S)2-hydroxy-(S)cyclohexylamino)-cyclohex-2-enone, and 3-((R)2-hydroxy-(R)cyclohexylamino)-cyclohex-2-enone.

Compounds of formula IX can be synthesized by various approaches. These approaches represent other aspects of the invention.

In one aspect, compounds of formula IX are synthesized by reacting a first compound having a 6-carbon cyclic structure comprising a keto group with a second compound having a 6-carbon cyclic structure, wherein either (a) the first compound comprises a primary amine group bonded to a cyclic carbon and the second compound comprises an oxygen bonded to a cyclic carbon that is reactive with the primary amine, or (ii) the second compound comprises a primary amine group bonded to a cyclic carbon and the first compound comprises an oxygen bonded to a cyclic carbon that is reactive with the primary amine.

For example, the synthesis can include the reaction of a compound of formula II: 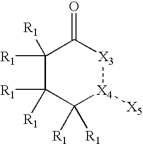
with a compound of formula III: 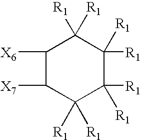
where, in both formula II and formula III, R₁ is independently selected from single and multi-atom groups, and either in formula II X₃—X₄—X₅ is C═C—NR₂H, wherein R₂ is a single or multi-atom group and in formula III X₆ and X₇ form an oxide ring; or in formula II X₃—X₄—X₅ is C—C═O and in formula III X₆ is OH and X₇ is NR₂H wherein R₂ is a single or multi-atom group.

In some preferred aspects, each R₁ and R₂ of formula II and III is independently selected from oxidatively non-reactive groups, and more preferably R₁ is selected from H and C₁-C₄ alkyl and R₂ is H or C₁-C₄ alkyl.

Compounds of formula II and formula III can be used to prepare 3-(2-hydroxy-cyclohexylamino)-cyclohex-2-enone. Enatiomeric forms of formula II and formula III can be utilized to prepare 3-(2-hydroxy-cyclohexylamino)-cyclohex-2-enone with the desired stereochemistry.

For example, in one mode of synthesis, 1,3 cyclohexanedione is reacted with 2-aminocyclohexanol to provide 3-(2-hydroxy-cyclohexylamino)-cyclohex-2-enone. 1,3 cyclohexanedione is commercially available from various sources, including for example, Sigma-Aldrich (St. Louis, Mo.) and Robinson Brothers Ltd. (West Bromwich, West Midlands, UK). The 2-aminocyclohexanol can be cis or trans, to provide the corresponding cyclohex-2-enone product in cis or trans configuration. (It is noted, however, that a product resulting from the oxidation and cyclization of the cyclohex-2-enone compound will result in the loss of the hydroxyl group and subsequently the chirality of the carbons on the cyclohexyl ring structure.) Cis or trans (or mixtures thereof) 2-aminocyclohexanol is commercially available from various sources, including for example, Sigma-Aldrich (St. Louis, Mo.) and Gentaur (Brussels, Belgium).

In one mode of practice compounds of Formula IX and formula II, such as 1,3 cyclohexanedione and 2-aminocyclohexanol, respectively, are dissolved in a non-polar aprotic solvent having a high boiling point, such as toluene. The compounds can be reacted at equimolar or near equimolar amounts. The compounds can be refluxed at temperatures of greater than 100° C., such as about 135° C. The product can be cooled and crystallized, followed by filtering, washing in a solvent such as toluene, and drying.

The reaction provides excellent yields of compounds of formula IX (e.g., 3-(2-hydroxy-cyclohexylamino)-cyclohex-2-enone), in the range of 95%-100%.

In another mode for the synthesis, 3-amino-2-cyclohexen-1-one is reacted with cyclohexene oxide to provide 3-(2-hydroxy-cyclohexylamino)-cyclohex-2-enone. 3-amino-2-cyclohexen-1-one is commercially available from various sources, including for example, ChemPur (Karlsruhe, Germany) and Lancaster Synthesis Inc (Windham, N.H.). Cyclohexene oxide is commercially available from Fluka (St. Louis, Mo.).

In another aspect, the invention provides compounds of formula X: 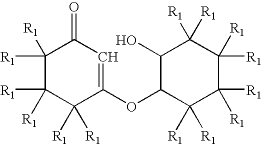
wherein R₁ is independently selected from single and multi-atom groups, and preferably oxidatively non-reactive groups, for example, H and C₁-C₄ alkyl groups.

Preferred compounds of the present invention include those wherein R₁ is H.

Exemplary compounds of formula X include 3-((R)2-hydroxy-cyclohexyloxy)-cyclohex-2-enone and 3-((S)2-hydroxy-cyclohexyloxy)-cyclohex-2-enone.

Compounds of formula X can be synthesized by reacting (a) a first compound comprising a 6-carbon cyclic moiety having no carbon-carbon double bonds and comprising two keto groups with a (b) a second compound having a 6-carbon cyclic moiety no carbon-carbon double bonds and comprising two hydroxyl groups bonded to cyclic carbons.

For example, the synthesis can include the reaction of a compound of formula IV: 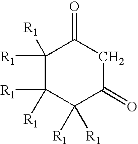
with a compound of formula V: 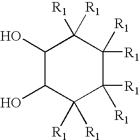
where, in both formula IV and formula V, R₁ is independently selected from single and multi-atom groups, and preferably oxidatively non-reactive groups, for example, H and C₁-C₄ alkyl groups.

An exemplary compound of formula IV is cyclohexanedione and an exemplary compound of formula V is trans or cis cyclohexanediol.

In some aspects, compounds of formula VIII are subjected to an oxidation and cyclization reaction that cause elimination of the —OH group from the second cyclic moiety (b) via a keto intermediate, and causing formation of a C—C bond between the C at position 2 of the first cyclic moiety (a) and the C at position 1 of the second cyclic moiety (b), forming a compound having fused tricyclic structure which can be represented by compounds of formula XI:

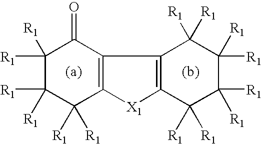

In providing structures of formula XI, and as indicated herein, R₁ is preferably individually selected from groups that are non-reactive under oxidative conditions leading to the formation of a compound of formula XI. Preferably, R₁ at any position is independently selected from the group consisting of H and linear or branched alkyl groups, such as C₁-C₄ linear or branched alkyl groups.

For example, oxidation and cyclization of compounds of formula IX, can cause formation of oxocarbazole compounds of formula XII:

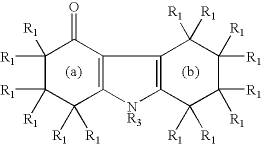

In another aspect, compounds of formula X are subjected to an oxidation and cyclization providing compounds of formula XIII:

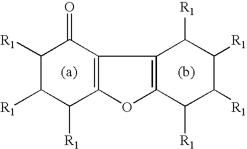

Oxidation can be performed in the presence of a halogenated aromatic hydrocarbon and a catalyst. Exemplary aromatic halogenated aromatic hydrocarbons include halotoluenes, such as bromotoluene. During the oxidation process, the oxidant, such as bromotoluene, is converted into a byproduct, such as toluene.

The catalyst can be chosen to complex with a portion of the oxidant during the oxidation process. One preferred catalyst comprises palladium, such as a palladium(0)-phosphine complex. An exemplary palladium catalyst is tetrakis(triphenylphosphine)palladium.

In one mode of practice, oxidation of the —OH group of compounds of formula VIII (c.f VIII) can be performed by combining a c.f VIII in the presence of a palladium catalyst, the halogenated aromatic hydrocarbon, and a base. Suitable bases include anyhydrous carbonate bases, such as anhydrous potassium carbonate.

In the catalytic cycle, oxidative addition of the halogenated aromatic hydrocarbon to the palladium catalyst occurs, forming a complex: 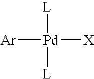
wherein L represents a ligand, such as a triphenylphosphine ligand, Ar represents an aryl group, and X represents a halogen atom. In the presence of the base, the hydroxyl group of a c.f VIII is oxidized to a keto intermediate. Oxidation to the keto intermediate occurs via exchange of the halogen atom X with the oxygen atom of the hydroxyl group of c.f VIII, B-hydride elimination then produces the c.f VIII-keto intermediate and an arylpalladium hydride (HPdL₂Ar). The palladium(0) catalyst can be regenerated by reductive elimination of the aryl moiety.

Cyclization of the c.f VIII-keto intermediate occurs via C—C bond formation between the C at position 2 of cyclic structure (a) and the C at position 2 of cyclic structure (b) followed by loss of water.

The oxidation reaction can be carried out in the presence of an aprotic solvent having a boiling point of greater than 100° C., such as DMF. In some modes of practice, the base can be used in molar excess, such as about a two-fold or greater molar excess in relation to the compound of formula VIII. In some modes of practice, the oxidant is used in a molar amount approximately equivalent to that of the molar amount of the compound of formula VIII. Generally, the catalyst is used in a molar amount of a fraction of that of the compound of formula I, and in some modes of practice, the catalyst is used in a molar amount of about 1/40 of the molar amount of the c.f VIII is used.

In some modes of practice, the oxidation and cyclization reaction is carried out at a temperature of greater than 100° C., and preferably greater than 125° C., such as about 150° C.

One advantage is seen in that formation of the compound having a fused tricyclic structure can be formed without the need for strong acids that are typically used in Fischer indole synthetic steps. Acids typically used in Fischer indole synthesis include polyphosphoric acid, HCl, and H₂SO₄. In Fischer indole synthesis these acids are typically used in a molar excess over the starting reagents. Therefore, synthesis of a compound of formula XI or XII can be performed in a non-acidic solvent.

In some cases, compounds of formula XIII can be converted to oxocarbazole compounds of formula X.

Some methods for the conversion of compounds of formula XIII to compounds of formula XII is by condensation of a compound of formula XIII with ammonia or a primary amine. Treatment of 4,5,6,7-tetrahydro-4-oxobenzofuran with a primary amine has been described as an alternative route to the formation of 4,5,6,7-tetrahydro-4-oxoindoles (U.S. Pat. No. 3,467,755). See also Stetter, H and Lauterback, R. (1962) Ann. 655, 20.

Following the condensation reaction, dehydrogenation as described herein can be carried out to provide a polycyclic phenol compound of formula XIV.

In some aspects, dehydrogenation of a compound of formula XII is performed to provide a compound of formula XIV:

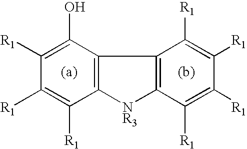

In a preferred aspect of the invention, catalytic dehydrogenation is performed. In some cases, catalytic dehydrogenation is desirable in order to provide a hydroxyl group on the cyclic moiety and avoid further oxidation of the hydroxyl group to a carboxylate group. Preferably, dehydrogenation is carried out using a catalyst selected from the group consisting of nickel, palladium, and platinum catalysts. A suitable catalyst is, for example, Raney nickel.

Dehydrogenation can be carried out in a solvent having a high boiling point, such as diphenyl ether. Preferably, the dehydrogenation reaction is carried out at a high temperature, such as above 200° C., and most preferably about 250° C. The reaction can be carried out for a suitable period of time, and can be analyzed by thin layer chromatography to determine the conversion of the carbazole to the hydroxycarbazole.

The reaction mixture can then be treated to purify the hydroxy carbazole product. For example, the reaction product can be diluted in an organic solvent, filtered, and then washed with an organic solvent. The organic washes can then be extracted with a basic solution, washed with an organic solvent, acidified, and then extracted. The organic extracts can then be combined, dried, filtered, and then concentrated.

Need to change the spec to reflect that the compounds used to provide 2-HCB are only used in the synthesis of the alkylhydroxamates.

Another aspect of the invention relates to methods for the synthesis of a compound of formula XV.

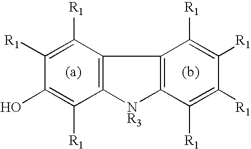

One compound of formula XV is 2-hydroxycarbazole (2-HCB) which is a useful intermediate compound in the synthesis of a tricyclic alkylhydroxamate as described in International Publication No. WO 02/085883 (see Example 3, 5, 6, 7, and 8).

Other synthetic approaches can be used to provide a compound of the invention comprising the bi-cyclic structure.

For example, a compound having the bi-cyclic structure can be formed in a process comprising the step of reacting (a) a first compound having a 6-carbon cyclic structure having at least one carbon-carbon double bond and comprising a hydroxyl group bonded to a cyclic carbon and an amine group bonded to a cyclic carbon with (b) a second compound having a 6-carbon cyclic structure having no carbon-carbon double bonds and comprising a reactive oxygen bonded to one or more cyclic carbons.

This step is preferably used for the synthesis of a compound of formula XV.

For example, the synthesis can include the reaction of a compound of formula VII: 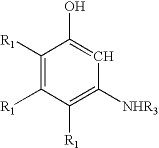
where, R₁ is independently selected from single and multi-atom groups, and preferably oxidatively non-reactive groups such as H and C₁-C₄ alkyl, and R₃ is a single and multi-atom groups, and preferably an oxidatively non-reactive group, such as H or C₁-C₄ alkyl, with a compound of formula III: 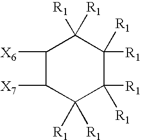
where R₁ is as defined herein, and X₆ and X₇ form an oxide ring.

The reaction of a compound of formula VII and a compound of formula III, as defined, results in the formation of a compound of formula VI.

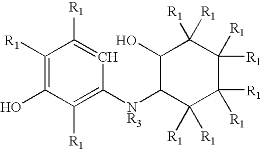

Further oxidation and cyclization of formula VI results in a compound of formula XVI:

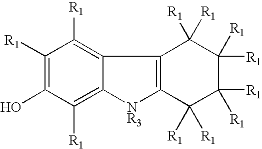

The compound of formula XVI can be subjected to dehydrogenation to provide a compound of formula XV.

In one mode for the synthesis of a compound of formula VI, 3-aminophenol is reacted with cyclohexene oxide to provide 3-(2-hydroxy-cyclohexylamino)-phenol. Oxidation, cyclization, and dehydration can be performed to provide 5,6,7,8-tetrahydro-carbazol-2-ol (CA#13314-79-9). Further dehydrogenation can provide 2-hydroxycarbazole.

The invention also provides methods for using compounds of the present invention for the synthesis of therapeutically useful compounds. In some aspects, a compound comprising the bi-cyclic structure described herein can be prepared as an intermediate compound in the synthesis of a therapeutically useful compound.

For example, a compound comprising a bi-cyclic structure of one of formula I, formula VIII, formula IX, formula X, or formula VI can be used as an intermediate compound for the synthesis of therapeutically useful compounds. In addition, precursors to any one of compounds of formula I, formula VIII, formula IX, formula X, or formula VI can be used in a synthetic scheme for the synthesis of therapeutically useful compounds. For example, one or more of a compound of formulas V, formula III, formula IV, formula V, or formula VII can be used for the preparation of an intermediate compound that is prepared in the synthesis of a therapeutically useful compound.

In some aspects, a compounds comprising a bi-cyclic structure of one of formula I, formula VIII, formula IX, or formula X can be used as intermediate compounds for the synthesis of a compound that has an affect on adrenergic receptors. The compound synthesized can be an adrenergic receptors antagonist or agonist. Compounds and methods of the invention can be employed to produce an adrenergic receptors antagonist that is used to treat hypertension. For example, the intermediate compound may be used in the synthesis of a beta blocker. Compounds and methods of the invention can be employed to produce an adrenergic receptor agonist used to treat type II diabetes and obesity.

In many aspects, the methods of the invention can be used to provide a carbazole, such as 4-hydroxycarbazole, which can then be reacted to provide a carbazole derivative. For example, the hydroxyl group of 4-hydroxycarbazole can be reacted with a chemical moiety to provide an oxycarbazole derivative.

In some aspects, a compound comprising a bi-cyclic structure of one of formula I, formula VIII, formula IX, or formula X can be used for the synthesis of carvedilol. Carvedilol is chemically named (1-carbazole-4-yloxy)-3-[2-(2-methoxyphenoxy)]ethylaminopropan-2-ol.

For example, any one of the following synthetic schemes can be applied for the production of carvedilol (wherein “c.f.” represents “compounds of formula” according to the invention, OHOC is 1,2,3,5,6,7,8,9-octahydro-4H-carbazol-4-one, 4-HCB is 4-hydroxycarbazole):
c.f. II+c.f. III→c.f.I→OHOC→4-HCB→carvedilol
c.f. II+c.f. III→c.f. IX→OHOC→4-HCB→carvedilol
c.f. IV +c.f. V→c.f. X→c.f. XIII→OHOC→4-HCB→carvedilol

The preparation of carvedilol from 4-HCB can be carried out by various approaches. Any approach described in the prior art utilizing 4-HCB for the synthesis of carvedilol can be used in conjunction with the methods described herein.

One approach involves reacting the hydroxyl group of 4-HCB with epichlorohydrin under basic conditions to provide 4-oxiranylmethoxy-9H-carbazole. The reaction can be performed in a polar organic solvent at a temperature in the range of 20° C.-100° C. 4-oxiranylmethoxy-9H-carbazole can then be reacted with benzyl-[2-(2-methoxyphenoxy] ethylamine in an organic solvent at a temperature in the range of 40° C.-140° C. Hydrogenation of the resulting compound results in loss of the benzyl group and formation of the final product carvedilol. The benzyl protected form of 2-(2-methoxyphenoxy) ethylamine reduces the amount of a bis impurity and increases carvedilol yeild. See EP Patent No. 918055.

Bis impurity reduction and increased carvedilol yeild can also be achieved by reacting an excess of 2-(2-methoxyphenoxy]ethylamine (unprotected) with 4-oxiranylmethoxy-9H-carbazole. Preferred molar ratios are in the range of 2.8:1 to 10:1. Preferred solvents for the reaction include toluene, xylene, and heptane. The reaction temperature can be in the range of 25° C. to 150° C., and more preferably in the range of 60° C. to 120° C. See U.S. Pat. No. 6,699,997.

In other aspects, a compounds comprising a bi-cyclic structure of one of formula I, formula VIII, formula IX, formula X, or formula VI can be used as intermediate compounds for the synthesis of a compound that has an affect on cell proliferation. For example, the compound synthesized using one of these intermediate compounds can be a tricyclic alkylhydroxamate. The compound synthesized can have histone deacylase inhibitor activity. Compounds and methods of the invention can be employed to produce a compound used to cancer.

Some therapeutically useful tricyclic alkylhydroxamates are described in International Publication No. WO 02/085883. Methods of the invention can be used to provide a carbazole, such as 4-hydroxycarbazole or 2-hydroxycarbazole, which can then be subjected to one or more other reaction conditions to provide a tricyclic alkylhydroxamate.

For example, any one of the following synthetic schemes can be applied for the production of carvedilol (2-HCB is 2-hydroxycarbazole):
c.f. II+c.f. III→c.f.I→OHOC→4-HCB→hydroxamide
c.f. II+c.f. III→c.f. IX→OHOC→4-HCB→hydroxamide
c.f. IV+c.f. V→c.f. X→c.f. XIII→OHOC→4-HCB→hydroxamide
c.f. VII+c.f. III→c.f. VI→c.f. XVI→2-HCB→hydroxamide

Other oxycarbazole derivatives are described in U.S. Pat. No. 6,140,352 which is directed to the synthesis of selective beta 3 adrenergic receptor agonists which can be used to treat diabetes. Exemplary oxycarbazole derivatives include (S)-4-[2-hydroxy-3-([4-(5-carbamoyl-2-pyridyloxy)phenyl]-2-methylpropylamino)propoxy]carbazole and (S)-4-[2-hydroxy-3-([4-(5-carboxy-2-pyridyloxy)phenyl]-2-methylpropylamino)propoxy]carbazole. For example, synthesis of the carbamoyl derivative can be accomplished by reacting 4-oxiranylmethoxy-9H-carbazole with 4-(2-amino-2-methylpropyl)phenoxy)-5-carboxamidepyridine in methanol at 60° C.

Some therapeutically useful carbazolone-based compounds include a substituent on the first cyclic moiety (e.g., the cyclohexenone moiety). For example, the anti-emetic drug odansetron (1,2,3,9-tetrahydro-9-methyl-3-[(2-methyl-1H-imidazol-1-yl)methyl]-4H-carbazol-4-one) has an imidazole-based group coupled to the cyclohexenone ring of the carbazolone portion of the molecule. Various approaches have been used for the synthesis of odansetron and are discussed in U.S. Patent Publication No. 2005/0020655.

### EXAMPLES

### Example 1

### 3-(2-Hydroxycyclohexyl)amino-2-cyclohexenone (Compound G)

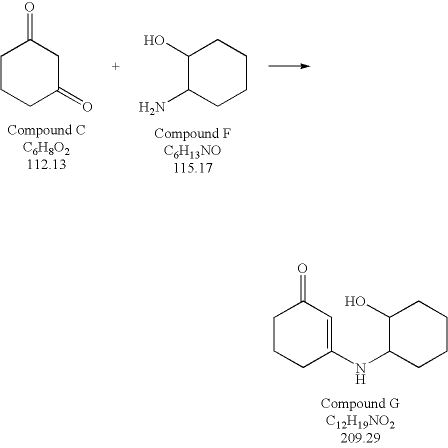

A 1000 mL 3-necked flask with teflon paddle stirrer/glass shaft, Dean-Stark trap with condenser and dry N₂ adapter, and a teflon stopper were used as equipment for the synthesis reaction.

A mixture of 20.43 g (176.8 mmol) of 97% cyclohexane-1,3-dione (compound C), 20.36 g (176.8 mmol) 2-aminocyclohexanol (compound F), and 300 mL toluene was refluxed (bath 135° C.) (Dean-Stark trap to collect H₂O; theoretical H₂O is 3.2 g, collected 2.9 mL) for 1.5 h. The suspension (2 layers) was cooled, resulting in crystallization of the lower layer. The solid was suction filtered, washed with 100 mL toluene, and dried in vacuo for 15.5 h to afford 36.41 g of bright yellow solid (NMR#5580).

Theoretical Yield—36.99 g

Percent Yield: 98.4% crude

### Example 2

### 1,2,3,5,6.7,8,9-Octahydro-4H-carbazol-4-one (OHOC; Compound I)

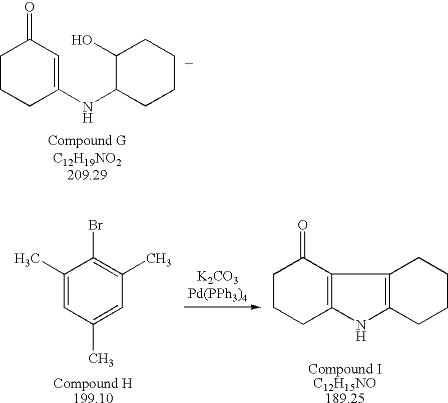

A 250 mL 3-necked flask with Teflon™ paddle stirrer, condenser with dry N2 adapter, and septum were used as equipment for the synthesis reaction

A mixture of 11.60 g (57.3 mmol) of 3-(2-hydroxycyclohexyl)amino-2-cyclohexenone_(Compound G) as described in Example 1, 8.9 mL (11.60 g, 58.3 mmol) 2-bromomesitylene (Compound H), 16.1 g (117 mmol) anhydrous potassium carbonate (K₂CO₃), 1.653 g (1.43 mmol) tetrakis(triphenylphosphine)palladium (Pd(PPh₃)₄), and 100 mL DMF was heated at 150° C. for 12 h. Pd(PPh₃)₄ [14421-01-3] mw is 1155.58 and was purchased from Strem (catalog #46-2150). The catalyst was stored in the freezer and handled in glove bag under N₂.

The solution was cooled and poured into 1000 mL H₂O. The mixture was extracted with ethyl ether five times (500 mL, 8×200 mL). The combined extracts were washed with 100 mL brine, dried (MgSO₄), filtered, and concentrated on a rotary evaporator at 25° C. and 60 mm Hg. The residue was taken up in 100 mL ethyl ether and the solid suction filtered, washed with 35 mL ethyl ether, and dried in vacuo (vacuum pump at 25° C. and 1 mm Hg for 19 h) to afford 8.16 g of beige solid. NMR was performed to confirm the identity of the compound.

Note: The ether mother liquors were concentrated in vacuo (rotary evaporator at 25° C. and 160 mm Hg then vacuum pump at 25° C. and 1 mm Hg for 47 h) to afford 2.19 g of yellow-brown tar-solid. NMR was performed to confirm the identity of the compound.

Theoretical Yield—10.85 g

Percent Yield: 75.2% crude

### Example 3

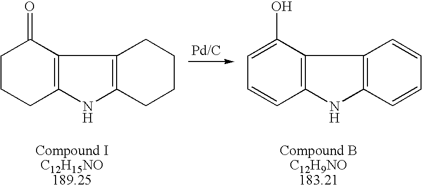

A 50 mL 3-necked flask with condenser (air cooling) with dry N₂ adapter, septum with stainless thermocouple, adapter with N₂ sparge tube (Ace Glass filter tube, porosity B, catalog #9436-04), mantle with Variac (set to 75%), aluminum foil/glass wool flask and jacket were used as equipment for the synthesis reaction

A suspension of crude OHOC (Compound 1) as prepared in Example 2 (0.500 g, 2.64 mmol) and 125 mg of 10% palladium on carbon (12.5 mg Pd, 0.118 mmol, 4.45 mol %) in 10 mL diphenyl ether was refluxed (pot temperature 250-255° C.).

To achieve a 250° C. pot, the sparge rate was reduced to 0.6 mL/min and the Variac setting increased to 80%. Heating was started at time 0 and at 18 minutes the pot temperature reached 200° C. At this time the sparge rate was reduced and at 35 minutes the pot temperature reached 250° C. After 5 h at 250° C., there was no detectable increase in conversion (low conversion). At this time 250 mg of additional Pd/C was added to the suspension (at 100° C.) which was then re-heated over 12 minutes to 250° C., and then kept at 250° C. for 1 h. After the 1 hour high conversion was seen as determined by a good Compound B/Compound I ratio. Product analysis was performed by TLC (7:3 hexanes-EtOAc), which separates the starting material, Compound B, Compound I, and diphenyl ether.

The suspension was cooled, diluted with 10 mL toluene, then suction filtered through 2.0 g of cellulose. The cellulose cake was washed with 10 mL toluene. The combined organic layers were extracted with 10 mL of 1 N NaOH twice. The (deep purple) aqueous layers were washed with 10 mL toluene three times, acidified with 21 mL of 1 N HCl, then extracted with 10 mL ethyl acetate three times. The combined organic extracts were dried (MgSO₄), filtered, and concentrated in vacuo (rotary evaporator at 35° C. and 70 mm Hg then vacuum pump at 25° C. and 1 mm Hg for 15 h) to afford 262.5 mg of dark solid.

The TLC of the ethyl acetate solution shows hydroxycarbazole and trace low Rf spot(s) which also darken in light/air.

Theoretical Yield=0.484 g

Percent Yield: 54.2% crude

### Example 4

### Cis- and trans-3-(2-Hydroxycyclohexyl)amino-2-cyclohexenone (Compound G)

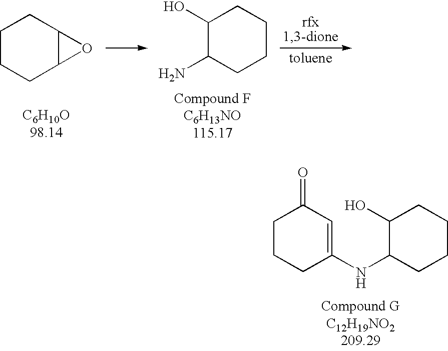

Trans-2-Aminocyclohexanol (trans-compound F) was prepared from a mixture of 10.0 mL (9.71 g, 98.9 mmol) of cyclohexene oxide, 68 mL (60.8 g, 17.0 g NH₃, 1.00 mol) of 28% ammonium hydroxide, and 40 mL methanol, which was stirred at 25° C. for 23 h. Volatiles were removed by distillation at atmospheric pressure. The residual peach-colored syrup was Kugelrohr distilled at 175° C. (oven) and 0.4 mm Hg to afford 8.50 g (74.6%) of (trans-compound F) colorless solid.

Cis-3-[(2-Hydroxycyclohexyl)amino]-2-cyclohexen-1-one (cis-compound G) [464-271] was prepared from a mixture of 8.03 g (69.5 mmol) of 97% cyclohexanedione, 8.00 g (69.5 mmol) of cis-2-aminocyclohexanol (cis-8), and 125 mL toluene, which was refluxed (bath 130° C.) using a Dean-Stark trap for 1 h. (Theoretical H₂O=1.25 mL, collected 1.10 mL). After cooling the suspension to 25° C., the precipitate was suction filtered, washed with 25 mL toluene, and dried in vacuo (at 25° C. and 1 mm Hg) to afford 14.13 g (97.1%) of (cis-compound G) as a bright yellow solid.

An analytical sample was prepared by recrystallization from acetonitrile, m.p. 150-154° C.; 500 MHz ¹H NMR (CDCl₃) δ 5.6 (br, 1H), 5.14 (s, 1H), 4.02 (m, 1H), 3.6 (br, 1H), 3.34-3.29 (m, 1H), 2.37-2.34 (m, 2H), 2.31-2.29 (m, 2H); 1.97-1.92 (m, 2H), 1.89-1.86 (m, 1H), 1.75-1.44 (m, 6H), 1.31-1.24 (m, 1H); 125 MHz ¹³C NMR (CDCl₃) δ 197.7, 164.6, 96.5, 67.5, 54.5, 36.5, 32.4, 30.3, 25.9, 24.3, 22.1, 19.6; IR (KBr) 3292, 3115, 2936, 1583, 1530, 1270, 1188, 1142, 1126, 986 cm⁻¹. Anal. Calcd for C₁₂H₁₉NO₂: C, 68.87; H, 9.15; N, 6.69. Found: C, 68.70; H, 9.20; N, 6.89.

Trans-3-[(2-Hydroxycyclohexyl)amino]-2-cyclohexen-1-one (trans-compound G) [464-260] was prepared from a mixture of 8.03 g (69.5 mmol) of 97% cyclohexanedione, 8.00 g (69.5 mmol) of trans-2-aminocyclohexanol (trans-8), and 150 mL toluene, which was refluxed (bath 135° C.) using a Dean-Stark trap for 1 h. (Theoretical H₂O=1.25 mL, collected 1.25 mL). After cooling the suspension to 25° C., the precipitate was suction filtered, washed with 25 mL toluene, and dried in vacuo (at 25° C. and 1 mm Hg) to afford 14.50 g (99.7%) of (trans-9) as a bright yellow solid.

An analytical sample was prepared by recrystallization from acetonitrile, m.p. 190-192° C.; 500 MHz ¹H NMR (CDCl₃) δ 5.22 (s, 1H), 5.1 (br, 1H), 3.45-3.40 (m, 1H), 3.19-3.13 (m, 1H), 2.41-2.24 (m, 4H), 2.15-2.1 (br, 1H), 2.08-2.06 (m, 1H), 1.97-1.92 (m, 2H), 1.78-1.76 (m, 1H), 1.70-1.67 (m, 1H), 1.43-1.22 (m, 3H), 1.11-1.05 (m, 1H); 125 MHz ¹³C NMR (CDCl₃) δ; IR (KBr) 3303, 3103, 2935, 2864, 1586, 1192, 1070, 808 cm⁻¹. Anal. Calcd for C₁₂H₁₉NO₂: C, 68.87; H, 9.15; N, 6.69. Found: C, 68.87; H, 9.19; N, 6.76.

A mixture of cis- and trans-3-[(2-Hydroxycyclohexyl)amino]-2-cyclohexen-1-one (compound G) [431-097] was prepared from a mixture of 20.43 g (176.8 mmol) of 97% cyclohexanedione, 20.36 g (176.8 mmol) of 2-aminocyclohexanol (compound F) (˜4:1 cis-trans mixture), and 300 mL toluene was refluxed (bath 135° C.) using a Dean-Stark trap for 1.5 h. (Theoretical H₂O=3.2 mL, collected 2.9 mL). The two-phase suspension was cooled, resulting in crystallization of the lower layer. The precipitate was suction filtered, washed with 100 mL toluene, and dried in vacuo (at 25° C. and 1 mm Hg) to afford 36.41 g (98.4%) of Compound G as a bright yellow solid.

### Example 5

### 1,2,3,5,6,7,8,9-Octahydro-4H-carbazol-4-one (OHOC; Compound I)

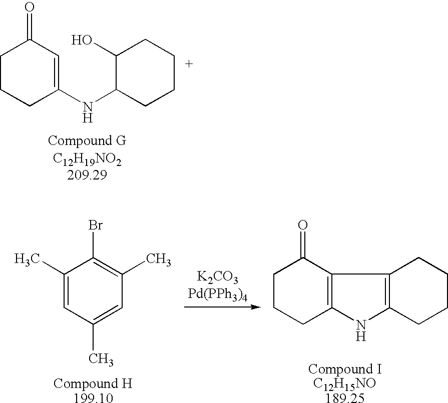

1,2,3,5,6,7,8,9-Octahydro-4H-carbazol-4-one (OHOC) (Compound I) [483-237] was prepared from a mixture of 1.179 g (5.83 mmol) of cis-3-[(2-hydroxycyclohexyl)amino]-2-cyclohexen-1-one (cis-Compound G), 0.89 mL (1.16 g, 5.83 mmol) 2-bromomesitylene (Compound H), 1.61 g (11.7 mmol) anhydrous potassium carbonate, 12.6 mg (0.0000109 mmol, 0.187 mol %) tetrakis(triphenylphosphine)palladium, and 1.0 mL DMF was heated at 150° C. for 15 h. The suspension was cooled and 10 mL H₂O added. The precipitate was suction filtered, washed with 10 mL H₂O, and air dried 2 h at 25° C. to afford 1.04 g (94.3%) of Compound I as a tan solid.

1,2,3,5,6,7,8,9-Octahydro-4H-carbazol-4-one (OHOC) (Compound I) [483-236] was prepared from a mixture of 1.179 g (5.83 mmol) of trans-3-[(2-hydroxycyclohexyl)amino]-2-cyclohexen-1-one (trans-Compound G), 0.89 mL (1.16 g, 5.83 mmol) 2-bromomesitylene, 1.61 g (11.7 mmol) anhydrous potassium carbonate, 13.3 mg (0.0000115 mmol, 0.197 mol %) tetrakis(triphenylphosphine)palladium, and 1.0 mL DMF was heated at 150° C. for 15 h. The suspension was cooled and 10 mL H₂O added. The precipitate was suction filtered, washed with 10 mL H₂O, and air dried 2.5 h at 25° C. to afford 0.98 g (89%) of Compound I as a tan solid.

1,2,3,5,6,7,8,9-Octahydro-4H-carbazol-4-one (OHOC) (Compound 1) [483-242] ] was prepared from a mixture of 1.179 g (5.83 mmol) of 3-[(2-hydroxycyclohexyl)amino]-2-cyclohexen-1-one (Compound G; cis-trans mixture), 0.89 mL (1.16 g, 5.83 mmol) 2-bromomesitylene, 1.61 g (11.7 mmol) anhydrous potassium carbonate, 14.9 mg (0.0000129 mmol, 0.221 mol %) tetrakis(triphenylphosphine)palladium, and 1.0 mL DMF was heated at 150° C. for 15 h. The suspension was cooled and 10 mL H₂O added. The precipitate was suction filtered, washed with 10 mL H₂O, and air dried 2.5 h at 25° C. to afford 0.99 g (90%) of Compound I as a tan solid.

### Example 6

### 4-Hydroxycarbazole

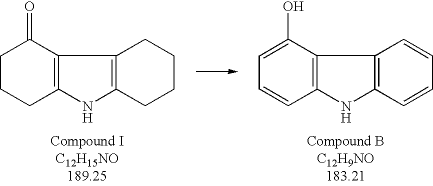

4-Hydroxycarbazole (Compound B) [475-010] was prepared by adding OHOC (compound I) (0.189 g, 1.00 mmol) to a solution of 0.330 g (5.00 mmol) of 85% potassium hydroxide pellets in 3 mL H₂O. Raney Nickel slurry (3.31 g) was added via disposable glass pipette and the resulting suspension refluxed under N₂ for 90 h.

The suspension was suction filtered through 2 g cellulose and the filter cake was washed with 10 mL H₂O. The combined mother liquors were acidified by adding 9.0 mL of 1 N HCl then extracted with 25 mL ethyl acetate. The combined extracts were dried (MgSO₄), filtered, and concentrated in vacuo (rotary evaporator at 35° C. and 65 mm Hg then vacuum pump at 25° C. and 1 mm Hg) to afford 0.140 g (76.9%) of Compound B as a colorless solid.

### Example 7

### 2-Hydroxycarbazole

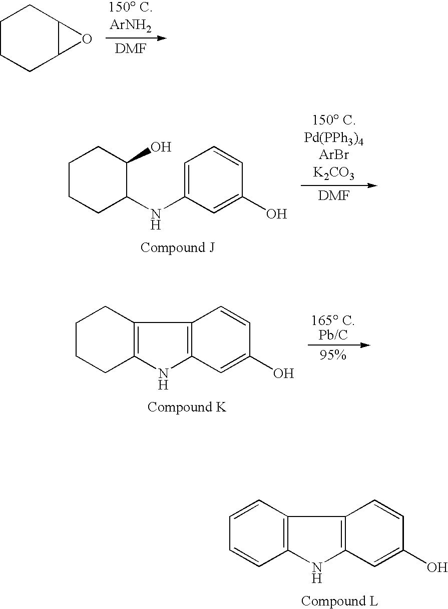

Cyclohexene oxide is converted to 2-hydroxycarbazole (Compound L) in three steps: ring opening with 3-aminophenol to produce aminoalcohol (Compound J), palladium-catalyzed oxidation/cyclization to 2-hydroxy-5,6,7,8-tetrahydrocarbazole (Compound K), and catalytic dehydrogenation. The uncatalyzed epoxide opening occurs at elevated temperature. The epoxide opening is catalyzed by Lewis acids.

What is claimed is:

1. A method for the synthesis of a carbazole or derivative thereof, comprising a step of preparing an intermediate compound comprising a bi-cyclic structure, the bicyclic structure comprising:
a 6-carbon first cyclic moiety comprising a keto group, or a hydroxyl group bonded to a cyclic carbon on the first cyclic moiety, and at least one carbon-carbon double bond that is present between alpha and beta cyclic carbons, and
a 6-carbon second cyclic moiety having no cyclic carbon-carbon double bonds and comprising a hydroxy group bonded to a cyclic carbon on the second cyclic moiety, wherein a cyclic carbon in the beta position on the first cyclic moiety is bonded via a divalent linking moiety to a cyclic carbon in the alpha position on the second cyclic moiety, and
a step of using the intermediate compound in a reaction for the synthesis of the carbazole or derivative thereof.

2. The method of claim 1, wherein the step of preparing the divalent linking moiety comprises an N or O atom.

3. The method of claim 1 wherein the step of preparing is used in a method for the synthesis of 4-hydroxycarbazole.

4. The method of claim 1 wherein the step of preparing is used in a method for the synthesis of a carbazole or derivative thereof that can affect an adrenergic receptor.

5. The method of claim 4 wherein the step of preparing is used in a method for the synthesis of a carbazole or derivative thereof that is an adrenergic receptor antagonist.

6. The method of claim 5 wherein the step of preparing is used in a method for the synthesis of carvedilol.

7. The method of claim 1 wherein the step of preparing provides a compound of formula I: 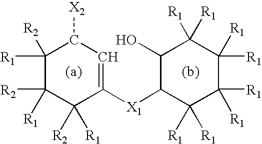
wherein X₁ is O or NR₃, and R₃ is a single or multi-atom group; and
C—X₂ is either C═O or C—OH,
wherein if C—X₂ is C═O then R₁ and R₂ are independently selected from single or multi-atom groups, or
if C—X₂ is C—OH then R₂ is zero and R₁ is independently selected from single and multi-atom groups, and
in the step of using, the compound of formula I is used in a reaction for the synthesis of the carbazole or derivative thereof.

8. The method of claim 7 wherein X₁ is NR₃.

9. The method of claim 8 wherein C—X₂ is C═O.

10. The method of claim 9 wherein each R₁ and R₂ are independently selected from oxidatively non-reactive groups.

11. The method of claim 10 wherein each R₁ and R₂ are independently selected from H and C₁-C₄ alkyl.

12. The method of claim 11 wherein the step of preparing provides 3-(2-hydroxy-cyclohexylamino)-cyclohex-2-enone:

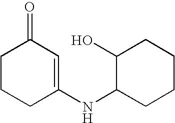

13. The method of claim 1 comprising a step of oxidation and cyclization of the intermediate compound to provide an oxocarbazole.

14. The method of claim 13 wherein the step of oxidation and cyclization comprises a palladium-catalyzed oxidation and cyclization of the intermediate compound.

15. The method of claim 14 wherein the step of oxidation and cyclization comprises an aryl-palladium alkoxide which mediates oxidation and cyclization of the intermediate compound.

16. The method of claim 13 comprising a step of palladium-catalyzed dehydrogenation of the oxocarbazole to provide a hydroxycarbazole.

17. The method of claim 1 wherein the step is used in a process to prepare 2-hydroxycarbazole.

18. The method of claim 1 wherein the step of preparing is used in a method for the synthesis of a carbazole or derivative thereof that can affect cell proliferation.

19. The method of claim 1 wherein the step of preparing is used in a method for the synthesis of a carbazole or derivative thereof that comprises histone deacylase inhibitor activity.

20. The method of claim 13 wherein the step of preparing is used in a method for the synthesis of a carbazole or derivative thereof that comprises a tricyclic alkylhydroxamate.

21. A compound of formula I: 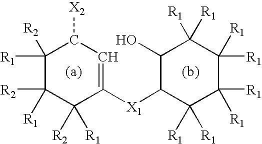
wherein X₁ comprises a divalent linking moiety; and
C—X₂ is either C═O or C—OH,
wherein if C—X₂ is C=O then each R₁ and R₂ are independently selected from single or multi-atom groups, or
if C—X₂ is C—OH then R₂ is zero and each R₁ is independently selected from single and multi-atom groups.

22. The compound of claim 21 wherein X₁ is O or NR₃, and R₃ is a single or multi-atom group.

23. The compound of claim 22 wherein C—X₂ is C═O.

24. The compound of claim 21 wherein each R₁ and R₂ are independently selected from oxidatively non-reactive groups.

25. The compound of claim 24 wherein each R₁ and R₂ are independently H or C₁-C₄ alkyl.

26. The compound of claim 25 which is 3-(2-hydroxy-cyclohexylamino)-cyclohex-2-enone:

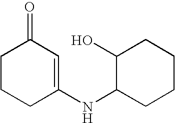

27. The compound of claim 21 wherein X₁ is O.

28. The compound of claim 27 wherein C—X₂ is C═O.

29. The compound of claim 28 wherein each R₁ and R₂ are independently H or C₁-C₄ alkyl.

30. The compound of claim 29 which is 3-(2-hydroxy-cyclohexyloxy)-cyclohex-2-enone:

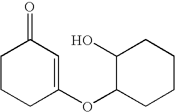

31. The compound of claim 21 wherein C—X₂ is C—OH.

32. The compound of claim 31 wherein R₂ is zero and each R₁ is independently H or C₁-C₄ alkyl.

33. The compound of claim 32 which is 3-(2-hydroxy-cyclohexylamino)-phenol:

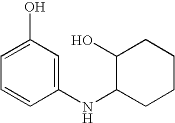

34. A method for the synthesis of a carbazole or derivative thereof, the method comprising:
a step of reacting:
a first compound comprising a 6-carbon cyclic structure comprising a keto group with,
a second compound comprising a 6-carbon cyclic structure, wherein either the first compound comprises a primary amine group bonded to a cyclic carbon and the second compound comprises an oxygen bonded to a cyclic carbon that is reactive with the primary amine of the first compound, or the second compound comprises a primary amine group bonded to a cyclic carbon and the first compound comprises an oxygen bonded to a cyclic carbon that is reactive with the primary amine of the second compound, to provide an intermediate compound comprising a bi-cyclic structure, and
a step of using the intermediate compound for the synthesis of a carbazole or derivative thereof.

35. The method of claim 34 wherein the step of reacting, the first compound is a compound of formula II: 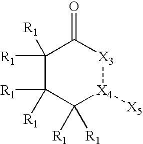
which is reacted with the second compound, which is a compound of formula III: 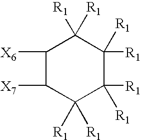
where, in both formula II and formula III, each R₁ is independently selected from single and multi-atom groups and either
in formula II X₃—X₄—X₅ is C═C—NR₂H, wherein R₂ is a single or multi-atom group and in formula III X₆ and X₇ form an oxide ring; or
in formula II X₃—X₄—X₅ is C—C═O and in formula III X₆ is OH and X₇ is NR₂H wherein R₂ is a single or multi-atom group.

36. The method of claim 35 wherein the step of reacting, in formula II X₃—X₄—X₅ is C—C═O, wherein R₂ is an oxidatively non-reactive group, and in formula III X₆ is OH and X₇ is NR₂H wherein R₂ is oxidatively non-reactive group.

37. The method of claim 36 wherein the step of reacting, in formula II R₂ is an H or C₁-C₄ alkyl, and in formula III R₂ is H or C₁-C₄ alkyl.

38. The method of claim 37 wherein the step of reacting the first compound is 1,3-cyclohexanedione.

39. The method of claim 37 wherein the step of reacting the second compound is 2-aminocyclohexanol.

40. The method of claim 35 wherein the step of reacting, in formula II X₃—X₄—X₅ is C═C—NR₂H, wherein R₂ is an oxidatively non-reactive group, and in formula III X₆ and X₇ form an oxide ring.

41. The method of claim 40 wherein the step of reacting, in formula II R₂ is H or C₁-C₄.

42. The method of claim 41 wherein the first compound is 3-amino-2-cyclohexene-1-one.

43. The method of claim 41 wherein the second compound is cyclohexene oxide.

44. A method for the synthesis of a carbazole or derivative thereof, the method comprising a
step of reacting:
a first compound comprising a 6-carbon cyclic moiety having no carbon-carbon double bonds and comprising two keto groups with
a second compound comprising a 6-carbon cyclic moiety having no carbon-carbon double bonds and comprising two hydroxyl groups bonded to cyclic carbons,
to provide and intermediate compound comprising a bi-cyclic structure, and
a step of using the intermediate compound for the synthesis of a carbazole or derivative thereof

45. The method of claim 44 wherein the step of reacting, the first compound is a compound of formula IV: 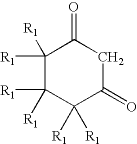
and the second compound is a compound of formula V: 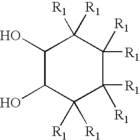
where, in both formula IV and formula V, each R₁ is independently selected from single and multi-atom groups.

46. The method of claim 45 wherein the first compound is cyclohexanedione.

47. The method of claim 45 wherein the second compound is cyclohexanediol.

48. A compound of formula VI: 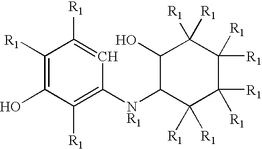
wherein each R₁ is independently selected from single and multi-atom groups.

49. The compound of claim 48 wherein each R₁ is independently selected from oxidatively non-reactive groups.

50. The compound of claim 48 wherein each R₁ is independently H or C₁-C₄ alkyl.

51. The compound of claim 50 which is 3-(2-Hydroxy-cyclohexylamino)-phenol:

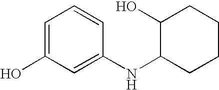

52. A method for the synthesis of a carbazole or derivative thereof, the method comprising:
a step of reacting
a first compound comprising a 6-carbon cyclic structure having at least one carbon-carbon double bond and comprising a hydroxyl group bonded to a cyclic carbon and an amine group bonded to a cyclic carbon with
a second compound comprising a 6-carbon cyclic structure having no carbon-carbon double bonds and comprising a reactive oxygen bonded to one or more cyclic carbons,
to provide an intermediate compound comprising a bi-cyclic structure, and
a step of using the intermediate compound for the synthesis of a carbazole or derivative thereof.

53. The method of claim 52 wherein the step of reacting comprises reacting the first compound which comprises a compound of formula VII: 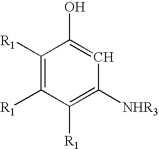
wherein each R₁ is independently selected from single and multi-atom groups and R₃ is a single or multi-atom group,
with the second compound which comprises a compound of formula III: 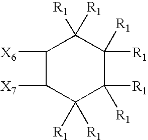
wherein each R₁ is independently selected from single and multi-atom groups and X₆ and X₇ form an oxide ring.

54. The method of claim 53 wherein the step of reacting comprises reacting the first compound comprising 3-amino-phenol.

55. The method of claim 53 wherein the step of reacting comprises reacting the second compound comprising cyclohexene oxide.

56. The method of claim 52, wherein the step of using the intermediate compound is for the synthesis of 2-hydroxycarbazole.
